(19) United States
(12) Patent Application Publication
(10) Pub. No.: US 2010/0240629 A1
(43) Pub. Date: Sep. 23, 2010

(54) POLYMORPHS OF FLUTICASONE FUROATE AND PROCESSES FOR PREPARATION THEREOF

(76) Inventors: Adrienne KOVACSNE-MEZEI, Debrecen (HU); Roman Gabriel, Olomouc (CZ); Alexandr Jegorov, Dobra Voda (CZ)

Correspondence Address: KENYON & KENYON LLP, ONE BROADWAY, NEW YORK, NY, 10004, US

(21) Appl. No.: 12/727,884

(22) Filed: Mar. 19, 2010

Related U.S. Application Data
(60) Provisional application No. 61/243,394, filed on Sep. 17, 2009.
(60) Provisional application No. 61/260,118, filed on Nov. 11, 2009.
(60) Provisional application No. 61/245,486, filed on Sep. 24, 2009.
(60) Provisional application No. 61/161,609, filed on Mar. 19, 2009.
(60) Provisional application No. 61/169,977, filed on Apr. 16, 2009.
(60) Provisional application No. 61/172,073, filed on Apr. 23, 2009.

(51) Int. Cl. A61K 31/58; C07D 307/68; A61P 11/06; A61P 17/06

(52) U.S. Cl. 514/172; 549/488

(57) ABSTRACT

The present invention provides crystalline forms of Fluticasone furoate, characterized by the data disclosed in the specification; pharmaceutical compositions comprising any one or combination of the crystalline forms of Fluticasone furoate and at least one pharmaceutically acceptable excipient; and the use of the crystalline forms of Fluticasone furoate in the preparation of pharmaceutical formulations.

This application claims the benefits of (a) U.S. Provisional Application Nos. 61/243,394, 61/245,486 and 61/260,118 filed Sep. 17, 2009, Sep. 24, 2009, Nov. 11, 2009, (b) U.S. Provisional Application Nos. 61/161,609, 61/169,977 and 61/172,073 filed Mar. 19, 2009, Apr. 16, 2009 and Apr. 23, 2009, (c) U.S. Non-Provisional application Ser. No. 12/462,782 filed Aug. 6, 2009, and (d) international patent application No. PCT/U.S.09/004,534 filed Aug. 6, 2009, the disclosures of which applications are incorporated by reference.

### FIELD OF THE INVENTION

The present invention relates to polymorphs of Fluticasone furoate, processes for preparing said polymorphs and pharmaceutical compositions thereof.

### BACKGROUND OF THE INVENTION

Fluticasone furoate, S-(fluoromethyl) (6S,8S,9R,10S,11S,13S,14S,16R,17R)-6,9-difluoro-11,17-dihydroxy-10,13,16-trimethyl-3-oxo-6,7,8,11,12,14,15,16-octa-hydrocyclopenta[α]phenanthrene-17-carbothioate, has the following structure:

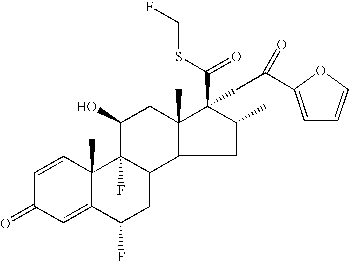

Fluticasone is a synthetic corticosteroid used for the treatment of asthma, allergic rhinitis. It can also be used in a cream or ointment for the treatment of eczema and psoriasis

Solvates of Fluticasone furoate are described in U.S. Pat. No. 7,101,866, U.S. Pat. No. 6,777,399, U.S. Pat. No. 6,777,400 and U.S. Pat. No. 6,858,593, incorporated herein by reference.

Polymorphism, the occurrence of different crystal forms, is a property of some molecules and molecular complexes. A single molecule may give rise to a variety of polymorphs having distinct crystal structures and physical properties like melting point, thermal behaviours (e.g. measured by thermogravimetric analysis—“TGA”, or differential scanning calorimetry—“DSC”), x-ray diffraction pattern, infrared absorption fingerprint, and solid state NMR spectrum. One or more of these techniques may be used to distinguish different polymorphic forms of a compound.

Discovering new polymorphic fauns and solvates of a pharmaceutical product can provide materials having desirable processing properties, such as ease of handling, ease of processing, storage stability, ease of purification or as desirable intermediate crystal forms that facilitate conversion to other polymorphic forms. New polymorphic fauns and solvates of a pharmaceutically useful compound can also provide an opportunity to improve the performance characteristics of a pharmaceutical product. It enlarges the repertoire of materials that a formulation scientist has available for formulation optimization, for example by providing a product with different properties, e.g., better processing or handling characteristics, improved dissolution profile, or improved shelf-life. For at least these reasons, there is a need for additional polymorphs of Fluticasone furoate.

### SUMMARY OF THE INVENTION

In one embodiment, the present invention encompasses crystalline Fluticasone furoate, designated form L, characterized by data selected from: a powder XRD pattern having peaks at 18.0°, 18.4°, 19.0°, 22.2° and 24.8°±0.2° 2θ; a PXRD pattern as depicted in FIG. 1; a solid state ¹³C NMR spectrum having peaks at 189.1, 165.5, 118.5 and 100.4±0.2 ppm; a solid-state ¹³C NMR spectrum having chemical shifts differences between the signal exhibiting the lowest chemical shift and another in the chemical shift range of 100 to 180 ppm of 88.7, 65.0 and 18.0±0.1 ppm; a solid state ¹³C NMR spectrum as depicted in FIG. 2; and any combination thereof.

In another embodiment, the present invention encompasses crystalline Fluticasone furoate, designated form M, characterized by data selected from: a powder XRD pattern having peaks at 12.0°, 12.4°, 17.6° and 21.8°±0.2° 2θ; a PXRD pattern depicted in FIG. 5; and any combination thereof.

In yet another embodiment the present invention encompasses the use of the above described polymorphs of Fluticasone furoate in the preparation of pharmaceutical formulations of Fluticasone furoate. In another embodiment, the present invention encompasses pharmaceutical compositions comprising any one, or combination, of the above described polymorphs of Fluticasone furoate and at least one pharmaceutically acceptable excipient.

### BRIEF DESCRIPTION OF THE FIGURES

FIG. 1 illustrates a powder X-ray diffraction pattern of crystalline Fluticasone furoate designated form L.

FIG. 2 illustrates a TGA pattern of crystalline Fluticasone furoate designated form L.

FIG. 3 illustrates a solid state ¹³C NMR spectrum of crystalline Fluticasone furoate designated form L.

FIG. 4 illustrates a crystal structure of Fluticasone furoate form L (S)-2-butanol solvate

FIG. 5 illustrates a powder X-ray diffraction pattern of crystalline Fluticasone furoate designated form M.

FIG. 6 illustrates a TGA pattern of crystalline Fluticasone furoate designated form M.

FIG. 7 illustrates a powder X-ray diffraction pattern of crystalline Fluticasone furoate designated form 1 according to U.S. Pat. No. 7,101,866.

FIG. 8 illustrates a powder X-ray diffraction pattern of crystalline Fluticasone furoate designated form N.

FIG. 9 illustrates a powder X-ray diffraction pattern of crystalline Fluticasone furoate designated form P.

FIG. 10 illustrates a powder X-ray diffraction pattern of crystalline Fluticasone furoate designated form R.

FIG. 11 illustrates a powder X-ray diffraction pattern of crystalline Fluticasone furoate designated form S.

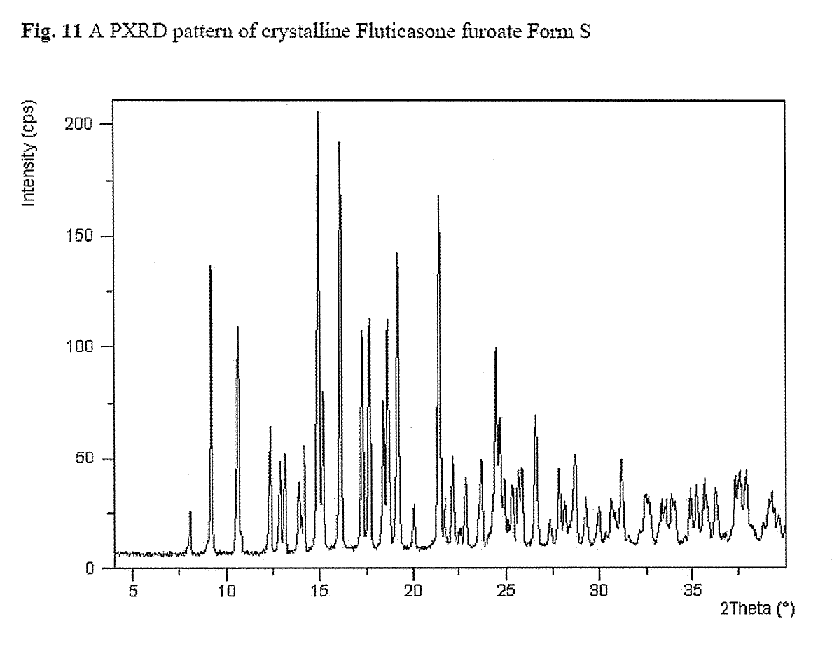

FIG. 12 illustrates a powder X-ray diffraction pattern of crystalline Fluticasone furoate designated form T.

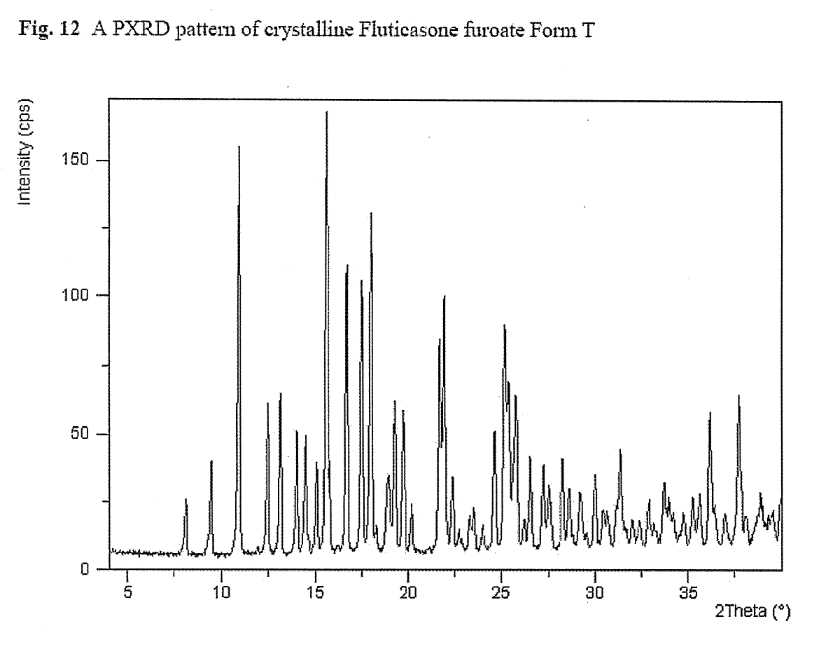

FIG. 13 illustrates a powder X-ray diffraction pattern of crystalline Fluticasone furoate designated form F obtained in example 7.

FIG. 14 illustrates a powder X-ray diffraction pattern of crystalline Fluticasone furoate designated form F obtained in example 8.

FIG. 15 illustrates a FT-IR pattern of crystalline Fluticasone furoate designated form F.

FIG. 16 illustrates a microscope image crystalline Fluticasone furoate designated form L.

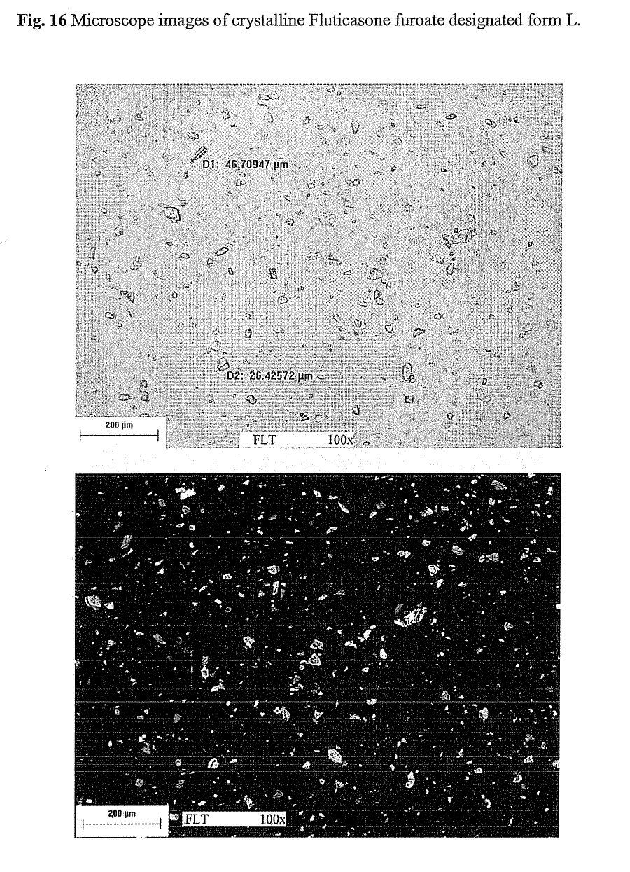

### DETAILED DESCRIPTION OF THE INVENTION

The present invention relates to polymorphs of Fluticasone furoate, processes for preparing said polymorphs, and pharmaceutical compositions thereof.

As used herein, the term “room temperature” refers to a temperature between about 20° C. and about 30° C., preferably about 20° C. to about 25° C.

As used herein, unless stated otherwise, XRPD peaks reported herein are preferably measured using CuK radiation, λ=1.54.

A crystal form may be referred to herein as being characterized by graphical data “as depicted in” a Figure. Such data include, for example, powder X-ray diffractograms and solid state NMR spectra. The skilled person will understand that such graphical representations of data may be subject to small variations, e.g., in peak relative intensities and peak positions due to factors such as variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms.

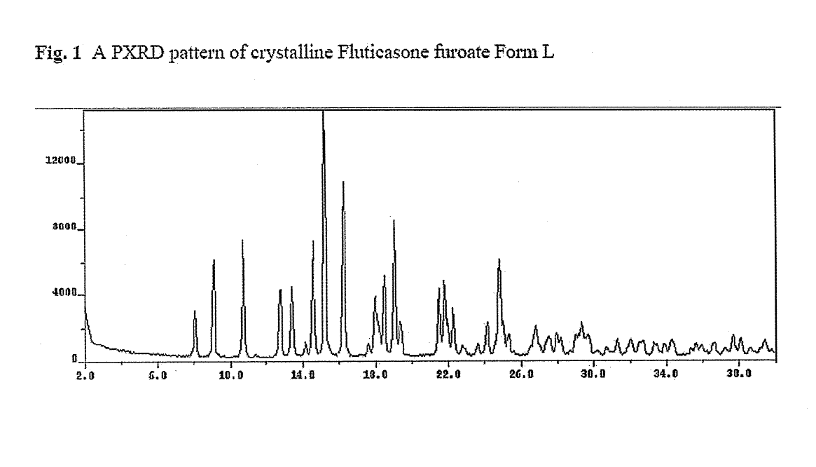

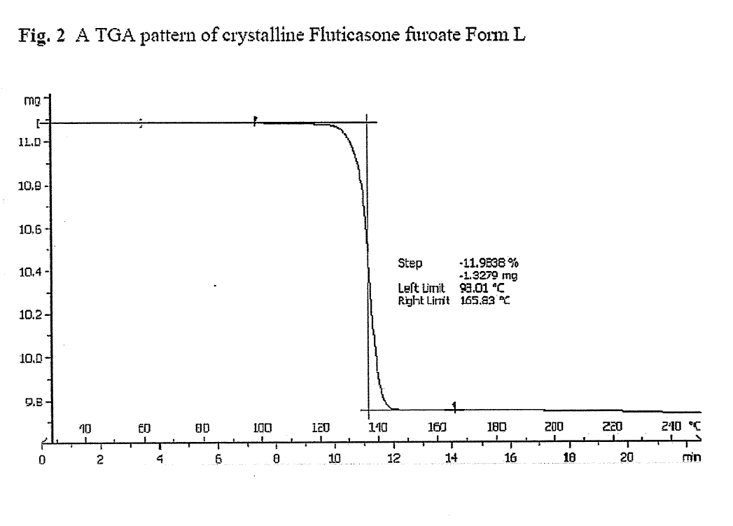

In one embodiment, the present invention encompasses crystalline Fluticasone furoate, designated form L, characterized by data selected from: a powder XRD pattern having peaks at 18.0°, 18.4°, 19.0°, 22.2° and 24.8°±0.2° 2θ; a PXRD pattern depicted in FIG. 1; a solid state ¹³C NMR spectrum having peaks at 189.1, 165.5, 118.5 and 100.4±0.2 ppm; a solid-state ¹³C NMR spectrum having chemical shifts differences between the signal exhibiting the lowest chemical shift and another in the chemical shift range of 100 to 180 ppm of 88.7, 65.0 and 18.0±0.1 ppm; a solid state ¹³C NMR spectrum as depicted in FIG. 2; and any combination thereof.

Typically, the signal exhibiting the lowest chemical shift in the chemical shift area of 100 to 180 ppm is at 100.4±1 ppm.

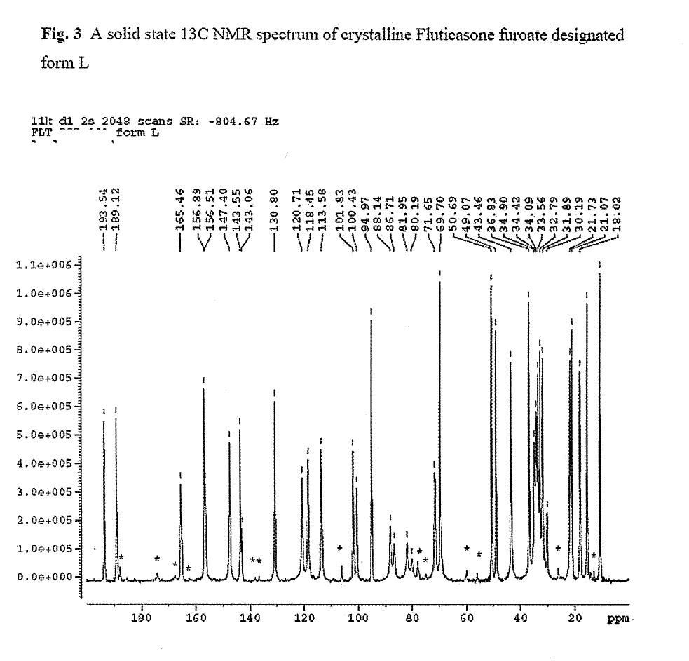

The above form L of Fluticasone furoate can be further characterized by data selected from: a powder XRD pattern having peaks at 9.0°, 10.7°, 14.5°, 15.2° and 16.2°±0.2° 2θ; a weight loss of up to about 12% at a temperature range of about 98° C. to about 166° C. as measured by TGA; a TGA pattern as depicted in FIG. 3; a solid state ¹³C NMR spectrum having peaks at 156.9, 147.4 and 120.7±0.2 ppm; a solid-state ¹³C NMR spectrum having chemical shifts differences between the signal exhibiting the lowest chemical shift and another in the chemical shift range of 100 to 180 ppm of 56.5, 47.0 and 20.3±0.1 ppm, and any combination thereof.

The weight loss of up to 12%, measured by TGA, corresponds to a theoretical 1:1 ratio of 2-butanol molecule vs. Fluticasone furoate molecule in the solvated form.

The above form L of Fluticasone furoate is a (S)-2-butanol solvate.

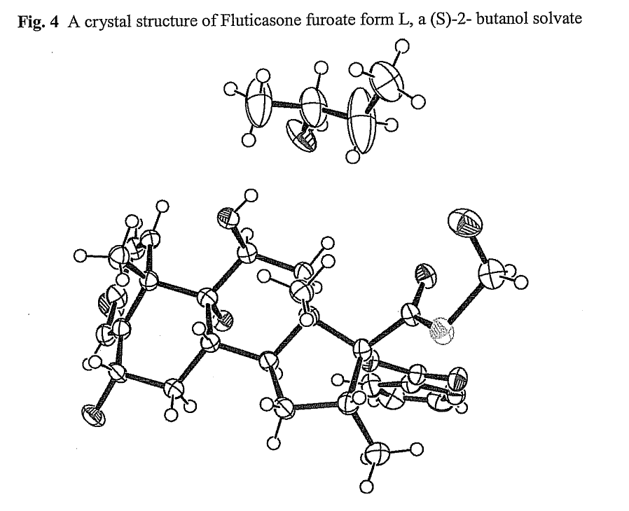

Alternatively, the above form L of Fluticasone furoate can be characterized by a crystal structure having the following unit cell parameters: α=12.3686(6) Å, b=15.4737(5) Å, c=15.5235(5) Å, α=90°, β=90°, λ=90°, cell volume=2971.0(2) Å³, orthorhombic space group P2₁2₁2₁. The above crystal structure is described in FIG. 4.

Form L of Fluticasone furoate has advantageous properties selected from at least one of: flowability, solubility, morphology or crystal habit, stability—such as storage stability, stability to dehydration, stability to polymorphic conversion and low hygroscopicity. Particularly, the crystalline Fluticasone furoate form L of the present invention has an irregular particle shape and small crystals with a particle size of less than 100 microns, qualities which provide the bulk product with excellent flowability properties that are of benefit for pharmaceutical formulations.

The above crystalline Fluticasone furoate form L is preferably substantially free of any other polymorph forms. As used herein the term “substantially free” refers to crystalline Fluticasone furoate form L containing 20% or less, 10% or less, 5% or less, 2% or less, and particularly, 1% or less, of any other or combination or other solid state forms of Fluticasone furoate, as measured by XRPD.

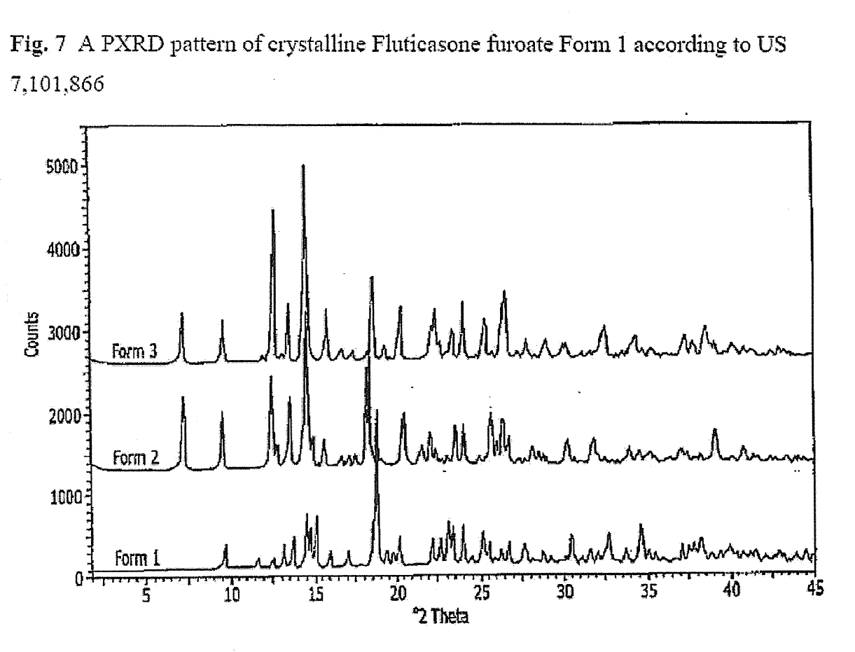

In particular, the above crystalline Fluticasone furoate form L is substantially free of anhydrous forms of Fluticasone furoate, designated form 1, form 2 and form 3, characterized by a PXRD pattern as depicted in FIG. 7. Typically, the amount of Fluticasone furoate anhydrous form 1 in the crystalline Fluticasone furoate form L of the present invention can be measured by PXRD using any peak from the group of peaks at about: 9.7, 11.6, 13.8, 20.2 and 23.1±0.2° 2θ; the amount of Fluticasone furoate anhydrous form 2 in the crystalline Fluticasone furoate form L can be measured by PXRD using any peak from the group of peaks at about: 7.2, 9.5, 12.4, 14.9 and 15.6±0.2° 2θ; and the amount of Fluticasone furoate anhydrous form 1 in the crystalline Fluticasone furoate form L can be measured by PXRD using any peak from the group of peaks at about: 7.2, 9.6, 15.8 and 20.2±0.2° 2θ.

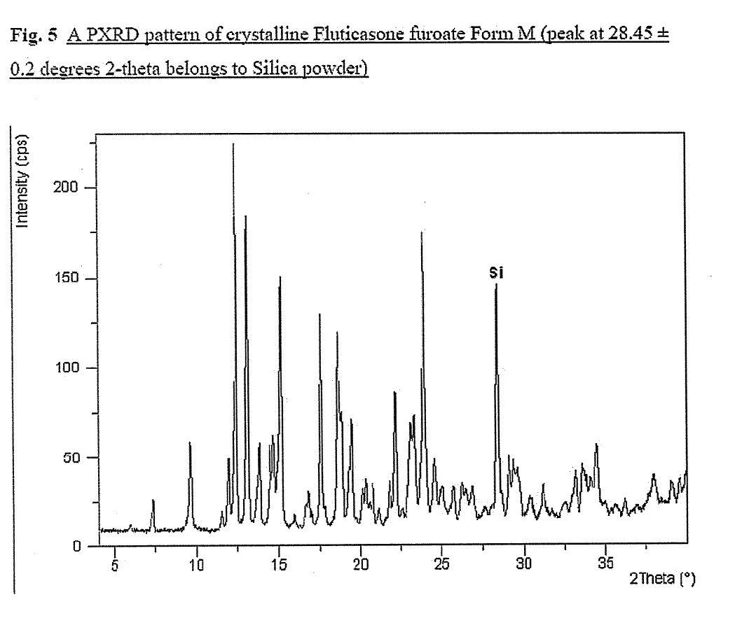

In another embodiment, the present invention encompasses crystalline Fluticasone furoate, designated form M, characterized by data selected from: a powder XRD pattern having peaks at 12.0°, 12.4°, 17.6° and 21.8°±0.2° 2θ; a PXRD pattern depicted in FIG. 5; and any combination thereof.

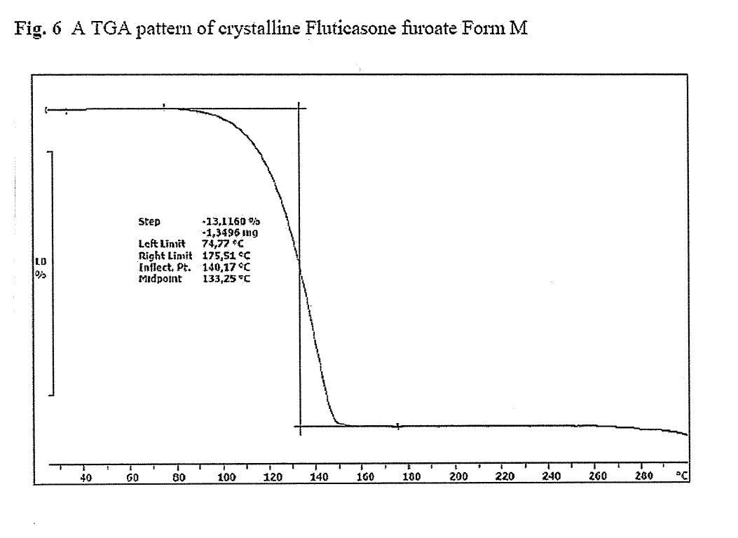

The above form M of Fluticasone furoate can be further characterized by data selected from: a powder XRD pattern having peaks at 13.1°, 15.2°, 18.6°, 18.9°, 19.5° and 23.9°±0.2° 2θ; a weight loss of up to about 13.1% at a temperature of about 25° C. about 175° C. as measured by TGA; a TGA pattern as depicted in FIG. 6; and any combination thereof.

Typically, the weight loss of up to about 13.1%, measured by TGA, corresponds to a theoretical 1:1 ratio of methylacetate molecule vs. Fluticasone furoate molecule in the solvated form.

The above form M of Fluticasone furoate is a methylacetate solvate.

Alternatively, the above form M of Fluticasone furoate can be characterized by a crystal structure having the following unit cell parameters: α=12.06129(13) Å, b=14.63587(14) Å, c=16.29340(17) Å, α=90°, β=90°, γ=90°, cell volume=2876.23(5) Å³, orthorhombic space group P 2₁2₁2₁.

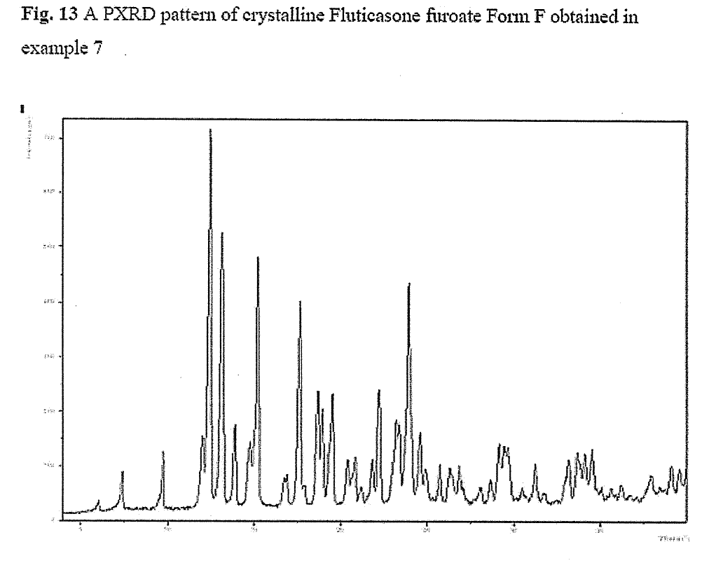

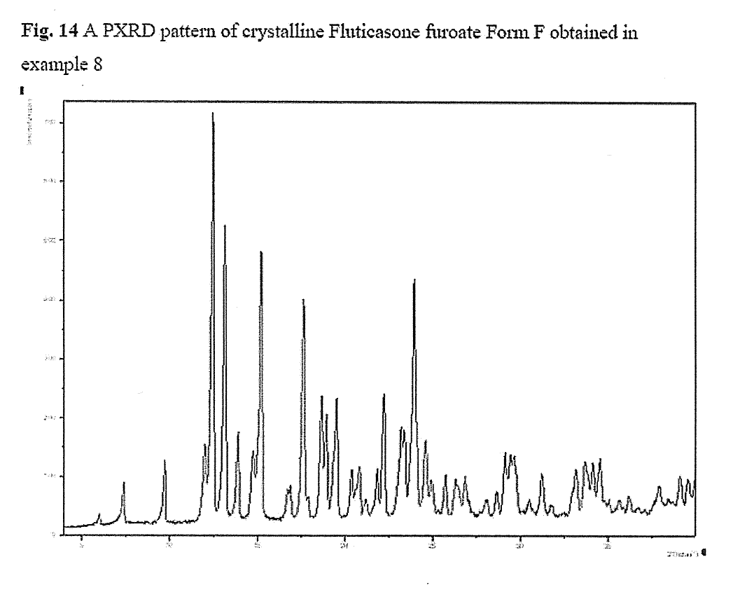

The present invention also describe crystalline form of Fluticasone furoate designated form F characterized by data selected from: powder XRD pattern having peaks at 7.4°, 12.5°, and 17.7°±0.2° 2θ, and any 2 peaks selected from a list consisting of: 13.2°, 15.3°, 18.7°, 19.6°, 22.3° and 24.0°±0.2° 2θ; a powder XRD pattern having peaks at 7.4°, 12.5°, 15.3°, 17.7° and 19.6°±0.2° 2θ; a PXRD pattern depicted in FIG. 13; a PXRD pattern depicted in FIG. 14; and any combination thereof.

The above form F of Fluticasone furoate is a 1,3 dimethylimidazolidinone (“DMI”) solvate.

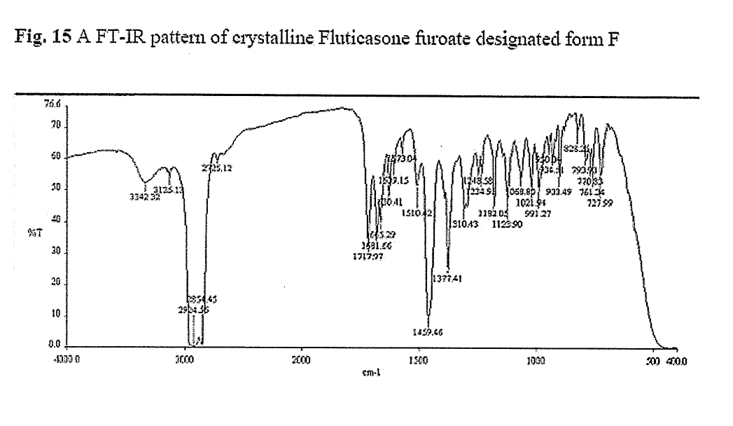

The above faun F of Fluticasone furoate can be further characterized by data selected from: a powder XRD pattern having peaks at 13.2°, 18.7°, 19.6°, 22.3° and 24.0°±0.2° 2θ; FT-IR pattern having peaks at about 3342, 1718, and 1682 cm⁻¹ and any 2 peaks selected from a list consisting of 1665, 1630, 1510, 1310, 1182, 1124 and 991 cm⁻¹; a FT-IR pattern depicted in FIG. 15; a content of DMI of 16.5% to about 21.8% by weight as measured by TGA and any combination thereof.

Alternatively, the above form F of Fluticasone furoate can be characterized by a crystal structure having the following unit cell parameters: α=30.4305(16) Å, b=7.5234(4) Å, c=14.705(1) Å, α=90°, β=105.587(5) °, γ=90 °, cell volume=3242.8(3) Å³, monoclinic space group C 2.

The present invention describes crystalline Fluticasone furoate characterized by data selected from at least one of: powder XRD pattern having peaks at 10.7°, 12.4°, 24.5° and 24.7°±0.2° 2θ; a PXRD pattern depicted in FIG. 6; and any combination thereof. This crystalline form of Fluticasone furoate can be designated form N.

The above form N of Fluticasone furoate can be further characterized by data selected from at least one of a powder XRD pattern having peaks at 15.0°, 16.2°, 17.3°, 17.7°, 18.7° and 21.4°±0.2° 2θ; a weight loss of up to about 16.7% at a temperature of about 25° C. to about 92° C. as measured by TGA; and any combination thereof.

Typically, the weight loss of up to about 16.7%, measured by TGA, corresponds to a theoretical 1:1 ratio of glycerol molecule vs. Fluticasone furoate molecule in the solvated form.

The above form N of Fluticasone furoate is a glycerol formal solvate.

The present invention also describes crystalline Fluticasone furoate characterized by data selected from at least one of: powder XRD pattern having peaks at 14.1°, 15.1°, 15.3°, 17.0° and 17.4°±0.2° 2θ; a PXRD pattern depicted in FIG. 7; and any combination thereof. This crystalline form of Fluticasone furoate can be designated form P.

The above form P of Fluticasone furoate can be further characterized by data selected from at least one of a powder XRD pattern having peaks at 12.2°, 21.2°, 21.4°, 24.5° and 25.6°±0.2° 2θ; a weight loss of up to about 13.5% at a temperature of about 27° C. to about 109° C. as measured by TGA; and any combination thereof.

Typically, the weight loss of up to about 16.5%, measured by TGA, corresponds to a theoretical 1:1 ratio of 2-methyl-tetrahydrofuran molecule vs. Fluticasone furoate molecule in the solvated form.

The above form P of Fluticasone furoate is a 2-methyl-tetrahydrofuran solvate.

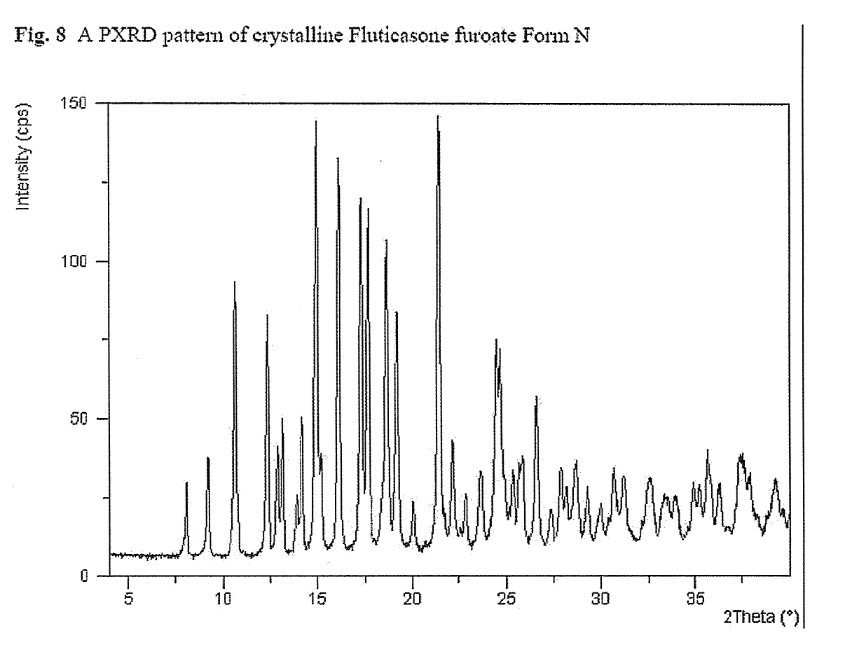

The present invention further describes crystalline Fluticasone furoate characterized by data selected from at least one of powder XRD pattern having peaks at 9.5°, 10.9°, 19.0° and 28.7°±0.2° 2θ; a PXRD pattern depicted in FIG. 8; and any combination thereof. This crystalline form of Fluticasone furoate can be designated form R.

The above form R of Fluticasone furoate can be further characterized by data selected from at least one of: a powder XRD pattern having peaks at 15.6°, 15.8°, 16.7°, 17.9°, 19.8° and 25.3°±0.2° 2θ; a weight loss of up to about 12.3% at a temperature of about 28° C. to about 147° C. as measured by TGA; and any combination thereof.

Typically, the weight loss of up to about 16.5%, measured by TGA, corresponds to a theoretical 1:1 ratio of dioxalane molecule vs. Fluticasone furoate molecule in the solvated form.

The above form R of Fluticasone furoate is a dioxolane solvate.

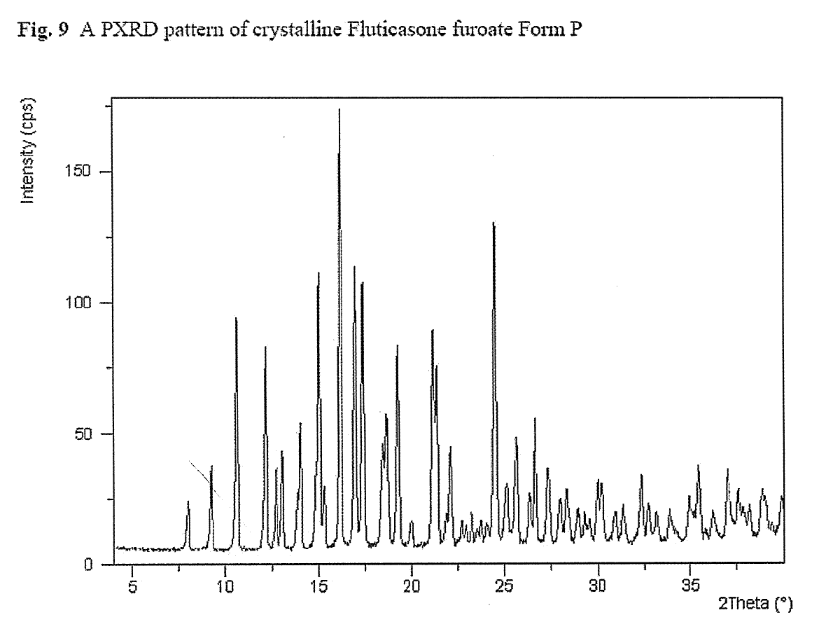

The present invention also describes crystalline Fluticasone furoate characterized by data selected from at least one of: powder XRD pattern having peaks at 9.2°, 18.5°, 18.7° and 19.2°±0.2° 2θ; a PXRD pattern depicted in FIG. 9; and any combination thereof. This crystalline form of Fluticasone furoate can be designated form S

The above form S of Fluticasone furoate can be further characterized by data selected from at least one of: a powder XRD pattern having peaks at 10.7°, 15.0°, 16.2°, 17.3°, 17.7° and 21.4°±0.2° 2θ; a weight loss of up to about 14.7% at a temperature of about 28° C. to about 143° C. as measured by TGA that corresponds to a theoretical 1:1 ratio of tetrahydropyran vs. Fluticasone furoate in the solvated form; and any combination thereof.

Typically, the weight loss of up to about 16.5%, measured by TGA, corresponds to a theoretical 1:1 ratio of tetrahydropyran molecule vs. Fluticasone furoate molecule in the solvated form.

The above foam S of Fluticasone furoate is a tetrahydropyran solvate.

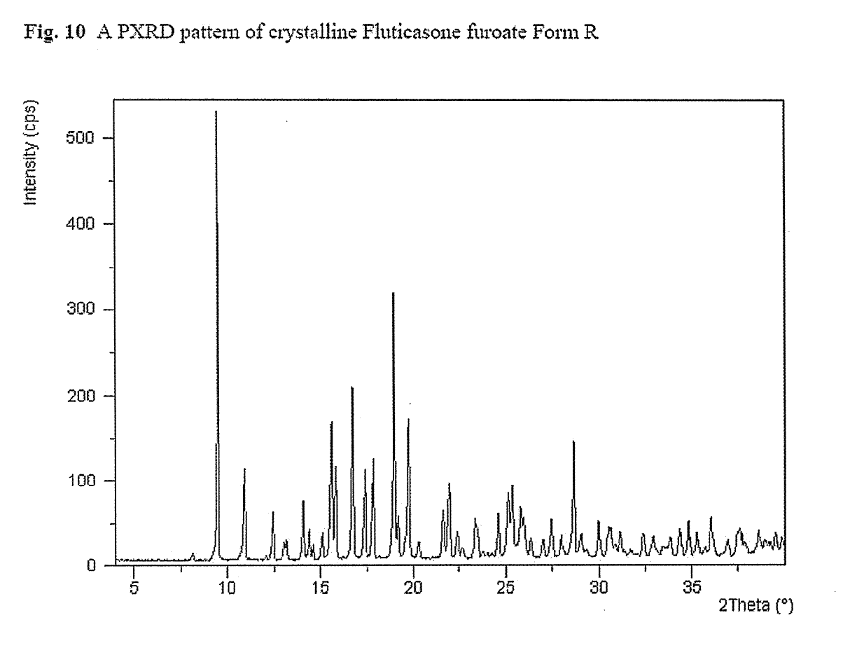

The present invention also describes crystalline Fluticasone furoate characterized by data selected from at least one of: powder XRD pattern having peaks at 13.2°, 17.5°, 18.0° and 26.5°±0.2° 2θ; a PXRD pattern depicted in FIG. 10; and any combination thereof. This crystalline Balm of Fluticasone furoate can be designated form T.

The above faun T of Fluticasone furoate can be further characterized by data selected from at least one of: a powder XRD pattern having peaks at 10.9°, 15.6°, 16.8°, 19.3°, 24.0° and 27.2°±0.2° 2θ; a weight loss of up to about 10.1% at a temperature of about 28° C. to about 143° C. as measured by TGA that corresponds to a theoretical 1:1 ratio of methylformate vs. Fluticasone furoate in the solvated form; and any combination thereof.

Typically, the weight loss of up to about 16.5%, measured by TGA, corresponds to a theoretical 1:1 ratio molecule of methylformate molecule vs. Fluticasone furoate in the solvated form.

The above form T of Fluticasone furoate is a methylformate solvate.

The above polymorphs of Fluticasone furoate can be used to prepare pharmaceutical compositions of Fluticasone furoate, by any method known in the art.

In one embodiment the present invention encompasses pharmaceutical compositions comprising any one, or combination, of the above described polymorphs of Fluticasone furoate and at least one pharmaceutically acceptable excipient.

### EXAMPLES

### A. XRD Analysis for Fluticasone Furoate Form L and F

The XRPD peaks referred to as characterizing form L throughout the invention and in the examples below, were obtained using an ARL X-ray powder diffractometer model X'TRA-030, Peltier detector, round standard aluminum sample holder with round zero background quartz plate was used. The cathode is CuKα radiation, λ=1.540562 Å. Scanning parameters: Range: 2-40° 2θ, continuous Scan, Rate: 3°/min. The accuracy of peak positions is defined as ±0.2° due to experimental differences like instrumentations and sample preparations.

### B. XRD Analysis for Fluticasone Furoate Forms M-T

The XRPD peaks referred to form M-T throughout the invention and in the examples below, were obtained using PanAnalytical X'pert with X'celerator detector diffractometer. The cathode is CuKα radiation, λ=1.540598 Å. The following parameters are required: continuous mode, spinning 16 rpm, step size 0.0167° and counting time 42 s, active length 2.122 mm, automatic divergence slits—automatic, irradiated length 10 mm, offset 0 mm, mask 10 mm, incident beam Soller slits 0.02 rad and diffracted beam Soller slits 0.04 rad.

The described peak positions of form M-T were determined by using a silicon powder as an internal standard in an admixture with the sample measured. The position of the silicon(Si) peak was corrected to silicone theoretical peak: 28.4409° two theta, and the positions of the measured peaks were corrected respectively. No correction was performed on the presented diffractograms in the figure.

### C. Thermal Gravimetric Analysis (TGA)

TGA/SDTA 851e, Mettler Toledo, Sample weight 7-15 mg.
Heating rate: 10° C./min., In N₂ stream: flow rate=50 ml/min
Scan range: Forms L and F: 30-250° C.; forms M-T: 25.0-300.0° C.

### D. FT-IR Spectroscopy

Perkin-Elmer Spectrum 1000 Spectrometer, at 4 cm⁻¹ resolution with 16 scans, in the range of 4000-400 cm⁻¹. Samples were analysed in Nujol mull. The spectra were recorded using an empty cell as a background

Perkin-Elmer Spectrum One Spectrometer, at 4 cm⁻¹ resolution with 16 scans, in the range of 4000-400 cm⁻¹. Samples were analysed in KBr with Drift technique. The spectra were recorded using KBr as a background.

### E. Single Crystal Analysis

Single crystal data were collected at 150 K (−123{tilde under (°)}) on Xcalibur PX, Cu Kα using combined φ and ω scans. Data collection, cell refinement, and data reduction were provided by program CrysAlisPro CCD. Programs SIR92 and Crystals were used to solve and refine the structure. All non-hydrogen atoms were refined anisotropically. The H atoms were all located in a difference Fourier's map, but those attached to carbon atoms were repositioned geometrically. 2-BuOH was found as a part of initial model obtained from direct methods.

### F. Solid State ¹³C NMR Analysis

Solid-state ¹³C NMR spectra were recorded with variable amplitude cross polarization, magic angle spinning and high power proton decoupling using a BRUKER Avance II+ spectrometer operating at 125 MHz and ambient temperature (about 25° C.—not controlled). A probe using 4 mm o.d. zirconia rotors was employed. The operation conditions were: contact time: 2 ms; recycle delay: 2 s; 2048 scans and spin rate of 11 kHz. Chemical shifts were referenced via a replacement sample of glycine (carboxyl carbon chemical shift assigned as 176.03 ppm relative to the signal of tetramethylsilane).

### Example 1

### Preparation of Fluticasone furoate form L, a (S)-2-butanol solvate

Fluticasone furoate dimethylacetamide(DMAc) solvate (1.0 g) was suspended in 2-butanol (commercially available racemic mixture, 20 ml). The suspension was stirred for 5 minutes at 22° C., then cooled over 10 minutes to 0° C. and stirred at 0° C. for 10 minutes. The cooled suspension was then heated up to 98° C. over 60 minutes and stirred at 98° C. for 10 minutes. The mixture was then cooled to 0° C. over 60 minutes and stirred at 0° C. for 60 minutes. The product was vacuum filtered and dried on the filter for 60 minutes under nitrogen. 0.9 g was obtained.

### Example 2

### Preparation of Fluticasone Furoate Form L, a (S)-2-Butanol Solvate

Fluticasone furoate DMAc solvate (400 mg) was dissolved in 2-butanol (commercially available racemic mixture, 50 ml) by heating to 100° C. for 3 min. Then the solution was allowed to cool in a box to 20° C. for about 12 hours, providing single crystals of fluticasone furoate (S)-2-butanol solvate. Suitable single crystal was directly mounted on the goniometer and cooled to 150 K (−123° C.) for the crystal structure determination: Unit cell parameters: α=12.3686(6) Å, b=15.4737(5) Å, c=15.5235(5) Å, α=90°, β=90°, γ=90°, cell volume=2971.0(2) Å³, orthorhombic space group P2₁2₁2₁. The Rietveld refinement of powder diffraction pattern of a laboratory sample at 20° C. provided unit cell parameters: α=12.68 Å, b=15.63 Å, c=15.74 Å, α=90°, β=90°, γ=90°, cell volume=2971.0 Å³, orthorhombic space group P2₁2₁2₁. The above crystal structure is described in FIG. 4.

### Example 3

### Preparation of Fluticasone Furoate Form L, a (S)-2-Butanol Solvate

Fluticasone furoate dimethylimidazolidinone (DMI) solvate (1.0 g) was suspended in 2-butanol (commercially available racemic mixture, 20 ml). The suspension was stirred for 5 minutes at 22° C., then cooled to 0° C. over 10 minutes, and stirred at 0° C. for 10 minutes. The cooled mixture was then heated up to 98° C. over 60 minutes and stirred at 98° C. for 10 minutes. The mixture was then cooled to 0° C. over 60 minutes and stirred at 0° C. for 60 minutes. The product was vacuum filtered for 60 minutes under nitrogen. 0.8 g was obtained.

### Example 4

### Preparation of Fluticasone Furoate Form L, a (S)-2-Butanol Solvate

300 mg of Fluticasone furoate DMI solvate (300 mg) was dissolved in 2-butanol (commercially available racemic mixture, 45 ml) by heating to 100° C. for 3 min. Then, the solution was put in a refrigerator set at −30° C. White crystals were formed within 10 hours at −30° C., and were recovered by filtration and air dried for 3 hours at 20° C.

### Example 5

### Preparation of Fluticasone Furoate DMAc Solvate According to U.S. Pat. No. 6,777,399 Example 15

6α,9α-Difluoro-17α-[(2-furanylcarbonyl)oxy]-11β-hydroxy-16α-methyl-3-oxo-androsta-1,4-diene-17β-carbothioic acid S-fluoromethyl ester (100 mg) was dissolved in dimethylacetamide (0.5 mL) at approximately 20° C. and left to slowly crystallize over a period of 6 days. The solid was recovered by filtration and then dried under vacuum at approximately 60° C. for 16 hours to afford the title compound. Stoichiometry of compound of formula (I): guest=1:1 from ¹H NMR (CDCl₃).

### Example 6

### Preparation of Fluticasone Furoate Form F, DMI Solvate

6α, 9α-Difluoro-17α-(2-furanylcarbonyl)oxy)-11β-hydroxy-16α-methyl-3-oxo-androsta-1,4-diene-17β-carbothionic acid (5.8 g), a Fluticasone furoate intermediate 1 and imidazole (0.9 g) were suspended in 60 ml of DMI. Bromofluoro-methane (2.4 g as 54% solution in DMI) was added and the resulting suspension was stirred at 40° C. for 15 min., then cooled at 30° C. for 30 mm. and then kept at 20° C. for 2 h: at 30° C. a solution was obtained. After reaction completion (judged by HPLC analysis), the mixture was warmed to 35° C. and 120 ml of water was slowly added over 1 hour, resulting in a suspension. The suspension was then cooled to 0° C. for 2 hours. The solid was filtered off, washed with water and dried at 60° C. under vacuum for 16 hours. Fluticasone furoate DMI solvate (6.10 g) was obtained. TGA result: 15.9%, PXRD: FIG. 14

### Example 7

### Preparation of Fluticasone Furoate Form F, DMI Solvate

Fluticasone furoate DMF solvate (0.8 g) was dissolved in 2.4 ml of 1.3 DMI at 80° C. The solution was cooled to room temperature and 12 ml of water was added. The resulting white suspension was stirred for 60 minutes at room temperature. The crystals are then isolated by filtration, washed with water, dried for 2 hours at 35° C. under nitrogen to provide a white solid (wet sample). TGA result: 21.8%, PXRD: FIG. 13.

### Example 8

### Preparation of Fluticasone Furoate Form F, DMI Solvate

A mixture of 0.5 g Fluticasone furoate DMAc solvate and 1.5 ml of DMI was dissolved at 25° C. The solution was then heated to 50° C. and 4.2 ml of water was added dropwise to form a white suspension. The suspension was stiller at 50° C. for 2.5 hours and then, white crystals were isolated by filtration and washed with water to provide the title compound. TGA result: 17.3%, PXRD: FIG. 14.

### Example 9

### Preparation of Fluticasone Furoate Form F, DMI Solvate

Flumethasone 17α-furoate-17β-thioacid triethylamine (TEA) salt (470 g, 0.773 mol) was suspended in 3260 ml 2-methyl tetrahydrofuran (2MeTHF), 56.4 ml water and 170 ml (1.21 mol) of TEA maintained at a temperature set at 23° C. BrCH₂F (108.26 g, 0.959 mol, 1.24 eq) dissolved in 375.5 g of dimethylacetamide (DMA) was added, maintaining the temperature in the range 20-25° C. After the addition was complete, the reaction mixture was stirred for 5 hours at 23° C. and then 16 hours at 0° C. Reaction completion was checked by HPLC, and then the mixture was warmed at 25° C. and 1400 ml of 2MeTHF and 1600 ml of water were added. The resulting biphasic mixture was stirred for 15 min. and then allowed to settle for 15 mm. The phases were separated. The organic phase was warmed to 35° C. and concentrated under vacuum to 1380 ml (3 vol., v/w) of residual volume at which point precipitation occurred. Another 1400 ml of 2MeTHF was added and the concentration was continued at the same temperature until 1380 ml (3 vol., v/w) of residual volume. DMI (2820 ml) was added to the suspension and the mixture was stirred until dissolution. The solution was concentrated under vacuum at 30° C. to 2820 ml (6 vol., v/w) of residual volume. While maintaining the temperature in the range 30-35° C., the DMI solution was diluted with aqueous ammonia (prepared by mixing 940 ml of 30% aqueous ammonia and 2350 ml of water). During the ammonia addition, a solid precipitate formed. The resulting suspension was stirred for 30 min at 30° C. and then cooled at −5-0° C. for 16 hours. The solid was collected by filtration, washed with 4700 ml of water and vacuum dried at 30° C. for 18 hours. Yield 449.2 g 89.0% molar yield of Fluticasone furoate DMI solvate

### Example 10

### Preparation of Fluticasone Furoate Form F, DMI Solvate

Fluticasone furoate (500 mg) was dissolved in 1,3-dimethyl-2-imidazolidinone (1.5 ml) at 80° C. The clear solution was left to crystallize over 8 days at 20° C. providing crystalline material. A single crystal was directly mounted on the goniometer and cooled to 150 K (−123° C.) for the crystal structure determination.

### Example 12

### Preparation of Fluticasone Furoate Dimethylformamide (“DMF”) Solvate According to U.S. Pat. No. 6,777,399, Example 5

A mixture of 6α,9α-Difluoro-17α-[(2-furanylcarbonyl)oxy]-11β-hydroxy-16α-methyl-3-oxo-androsta-1,4-diene-17β-carbothioic acid (4.5 g, 8.88 mmol) in DMF (31 ml) is treated with potassium bicarbonate (0.89 g, 8.88 mmol) and the mixture is cooled to −20° C. A solution of bromofluoromethane (0.95 g, 8.50 mmol, 0.98 eq) in DMF (4.8 ml) at 0° C. is added and the mixture is stirred at −20° C. for 4 hours. The mixture is then stirred at −20° C. for a further 30 minutes, added to 2M hydrochloric acid (100 ml) and stirred for a further 30 minutes at 0-5° C. The precipitate collected by vacuum filtration, washed with water and dried at 50° C. to give the title compound (4.47 g, 82%). NMR δ((CD₃OD) includes the peaks described for 6α,9α-difluoro-17α-[(2-furanylcarbonyl)oxy]-11β-hydroxy-16α-methyl-3-oxo-androsta-1,4-diene-17β-carbothioic acid S-fluoromethyl ester and the following additional solvent peaks: 7.98 (1H, bs), 2.99 (3H, s), 2.86 (3H, s).

### Example 13

### Preparation of Fluticasone Furoate Form M, Methylacetate Solvate

Fluticasone furoate (300 mg, Form 1, prepared according to example 16) was dissolved in methylacetate (4 ml) by heating at 56° C. for 3 minutes. The solution was allowed to cool to 20° C. and then allowed to evaporate spontaneously in an open flask to a residual volume about 1 ml. The mother liquor was filtered off and the residual crystalline material was allowed to air dry at 20° C. for 2 hours.

### Example 14

### Preparation of Fluticasone Furoate Form M, Methylacetate Solvate

Fluticasone furoate (750 mg, DMI solvate) was dissolved in methylacetate (10 ml) by heating at 56° C. for 3 minutes. The solution was allowed to cool to 20° C. and to evaporate spontaneously in an open flask to a residual volume about 3 ml. The mother liquor was filtered off and the remaining crystalline material was allowed to air dry at 20° C. for 2 hours. The sample was heated then at 80° C. and 2 mBar for 1 h.

### Example 15

### Preparation of Fluticasone Furoate Form M, Methylacetate Solvate

Fluticasone furoate (526 mg) was dissolved in methylacetate (15 ml) at 56° C. The resulting clear solution was left to crystallize for 5 days at 20° C. in an open flask providing crystalline material. A single crystal was directly mounted on the goniometer and cooled to 150 K (−123° C.) for the crystal structure determination.

### Example 16

### Preparation of Starting Material Fluticasone Furoate Form 1 (PXRD in FIG. 7) According to U.S. Pat. No. 7,101,866 Example 1

A suspension of 6α, 9α-Difluoro-17α-(2-furanylcarbonyl)oxy)-11β-hydroxy-16α-methyl-3-oxo-androsta-1,4-diene-17β-carbothionic acid, a Fluticasone furoate intermediate 1 (2.5 g, 4.94 mmol) was dissolved in anhydrous N,N-dimethylformamide (25 ml) and sodium hydrogen carbonate (465 mg, 5.53 mmol) was added. The mixture was stirred at −20° C. and bromofluoromethane (0.77 ml, 6.37 mmol) was added and the mixture was stirred at −20° C. for 2 h. Diethylamine (2.57 ml, 24.7 mmole) was added and the mixture stirred at −20° C. for 30 min. The mixture was added to 2M hydrochloric acid (93 ml) and stirred for 30 min. Water (300 ml) was added and the precipitate was collected by filtration, washed with water and dried in vacuo at 50° C. to give a white solid which was recrystallised from acetone/water (to yield the acetone solvate of 6α, 9α-difluoro-17α-[(2-furanylcarbonyl)oxy]-11β-hydroxy-16α-methyl-3-oxo-androsta-1,4-diene-17β-carbothioic acid S-fluoromethyl ester) and dried in vacuo at 50° C. to give the title compound (2.351 g, 88%): LCMS retention time 3.66 min, m/z 539 MH+, NMR δ (CDCl₃) includes 7.60 (1H, m), 7.18-7.11 (2H, m), 6.52 (111, dd, J=4.2 Hz), 6.46 (1H, s), 6.41 (1H, dd, J=10.2 Hz), 5.95 and 5.82 (2H dd, J 51, 9 Hz), 5.48 and 5.35 (1H, 2m), 4.48 (1H, m), 3.48 (1H, m), 1.55 (3H, s), 1.16 (3H, s), 1.06 (3H, d, J=7 Hz).

### Example 17

### Preparation of Fluticasone Furoate Form N, Glycerol Formal Solvate

Fluticasone furoate (200 mg, DMI solvate) was dissolved in glycerol formal (3 ml). The solution was cooled to 0° C. and water (0.75 ml) was added under stirring. The resulting solution was maintained overnight at −30° C. A solid was recovered by filtration, washed with water (10 ml) and allowed to dry at 20° C. for 5 h.

### Example 18

### Preparation of Fluticasone Furoate Form P, 2-Methyl-Tetrahydrofuran Solvate

Fluticasone furoate (form 1, 200 mg) was stirred in a suspension of 2-methyl-tetrahydrofuran (3 ml) at 20° C. for 80 min. Crystals formed and were recovered by filtration, and air dried at 20° C. for 3 h.

### Example 19

### Preparation of Fluticasone Furoate Form R, Dioxolane Solvate

Fluticasone furoate (300 mg, Form I) was dissolved in dioxolane (4 ml) by heating at 75° C. for 5 min. The resulting solution was allowed to cool to 20° C. over 15 min. Within 3 h, fluticasone furoate crystallized in the form of large white crystals. The crystals were recovered by filtration, washed with t-butyl methyl ether (10 ml) and air dried at 20° C. for 3 h.

### Example 20

### Preparation of Fluticasone Furoate Form S, Tetrahydropyran Solvate

Fluticasone furoate (300 mg, Form I) was dissolved in tetrahydropyran (8 ml) by heating at 88° C. for 5 min. The solution was allowed to cool to 20° C. over 15 min. Within 3 h fluticasone furoate crystallized in the form of large white crystals. The crystals were recovered by filtration, washed with t-butyl methyl ether (10 ml) and air dried at 20° C. for 3 h.

### Example 21

### Preparation of Fluticasone Furoate Form T, Methylformate Solvate

A suspension of fluticasone furoate (274 mg, Form I) in methylformate (13 ml) was stirred for 12 h at 20° C. to obtain a solid. The solid was recovered by filtration and air dried at 20° C. for 3 h.

### Example 22

### Preparation of Fluticasone Furoate Form T, Methylformate Solvate

A suspension of fluticasone furotate (600 mg, DMI solvate) in methyl formate (20 ml) was stirred for 12 h at 20° C. to obtain a solid. The solid was recovered by filtration, air dried at 20° C. for 3 h and at 50° C., 200 Pa for 2 h.

### Example 23

### Preparation of Fluticasone Furoate Form L, (S)-2-Butanol Solvate

Fluticasone furoate DMAc solvate (3.0 g) was suspended in 60 ml of 2-butanol (commercially available racemic mixture). The suspension was cooled over 10 minutes to 0° C. and stirred at 0° C. for 10 minutes. Then it was heated up to 98° C. over 60 minutes and stirred at 98° C. for 15 minutes. Then it was cooled to 0° C. over 60 minutes and stirred at 0° C. for 60 minutes. The product was filtered, washed with 2-butanol and dried for 60 minutes under vacuum and nitrogen. Yield: 2.7 g of Fluticasone furoate, form L.

### Example 24

### Preparation of Fluticasone Furoate Form L, (S)-2-Butanol Solvate

Fluticasone furoate DMAc solvate 5(0.0 g) was suspended in 100 ml of 2-butanol (commercially available racemic mixture). The suspension was cooled over 10 minutes to 0° C. and stirred at 0° C. for 10 minutes. Then it was heated up to 98° C. over 60 minutes and stirred at 98° C. for 15 minutes. Then it was cooled to 0° C. over 60 minutes and stirred at 0° C. for 60 minutes. The product was filtered, washed with 2-butanol and dried for 60 minutes under vacuum and nitrogen. Yield: 4.5 g of Fluticasone furoate form L.

### Example 25

### Preparation of Fluticasone Furoate Form L, (S)-2-Butanol Solvate

Fluticasone furoate DMI solvate (5.0 g) was suspended in 50 ml of 2-butanol (commercially available racemic mixture). The suspension was heated and refluxed (T=98° C.) for 30 minutes, then cooled at 0-5° C. in about 30 minutes. The suspension was kept at 0-5° C. for about 2 hours. A solid product was separated by filtration, washed with two 5 ml portions of 2-butanol and dried at 40° C. for 16 hours.

Yield: 4.3 g of Fluticasone furoate form L.

### Example 26

### Preparation of Fluticasone Furoate Form L, (S)-2-Butanol Solvate

Fluticasone furoate DMI solvate (4.0 g) was suspended in 43 ml of 2MeTHF and heated at 40-45° C. until dissolution. The solution was cooled to about 30° C. and then concentrated under vacuum until the volume was reduced to about 20 ml. 2-Butanol (20 ml) was added and the mixture was concentrated under vacuum for about 30 min, then at atmospheric pressure until internal temperature higher than 90° C. (T=93-98° C.). The suspension was diluted with 20 ml of 2-butanol (commercially available racemic mixture) and then heated at reflux (T=93-98° C.) for 30-60 min. The mixture was then cooled to 0° C. over about 30 min, kept cold for 2 hours, then filtered and washed with two 5 ml portions of 2-butanol. The solid was dried under vacuum at 40° C. for 16 hours. Yield: 3.4 g of Fluticasone furoate form L.

Invention claimed:

1. Crystalline Fluticasone furoate characterized by data selected from: a powder XRD pattern having peaks at 18.0°, 18.4°, 19.0°, 22.2° and 24.8°±0.2° 2θ; a PXRD pattern as depicted in FIG. 1; a solid state ¹³C NMR spectrum having peaks at 189.1, 165.5, 118.5 and 100.4±0.2 ppm; a solid-state ¹³C NMR spectrum having chemical shifts differences between the signal exhibiting the lowest chemical shift and another in the chemical shift range of 100 to 180 ppm of 88.7, 65.0 and 18.0±0.1 ppm; a solid state ¹³C NMR spectrum as depicted in FIG. 2; crystal structure having the following unit cell parameters: α=12.3686(6) Å, b=15.4737(5) Å, c=15.5235(5) Å, α=90°, β=90 °, γ=90°, cell volume=2971.0(2) Å³, orthorhombic space group P2₁2₁2₁; a crystal structure as shown in FIG. 4; and any combination thereof.

2. The crystalline Fluticasone furoate of claim 1, characterized by data selected from a group consisting of: a powder XRD pattern having peaks at 9.0°, 10.7°, 14.5°, 15.2° and 16.2°±0.2° 2θ; a solid state ¹³C NMR spectrum having peaks at 156.9, 147.4 and 120.7±0.2 ppm; a solid-state ¹³C NMR spectrum having chemical shifts differences between the signal exhibiting the lowest chemical shift and another in the chemical shift range of 100 to 180 ppm of 56.5, 47.0 and 20.3±0.1 ppm; a weight loss of up to about 12% at a temperature range of about 98° C. to about 166° C. as measured by TGA; a TGA pattern as depicted in FIG. 3; and any combination thereof.

3. The crystalline Fluticasone furoate of claim 1, wherein the crystalline Fluticasone furoate is a (S)-2-butanol solvate.

4. The crystalline Fluticasone furoate of claim 1, characterized by a powder XRD pattern having peaks at 18.0°, 18.4°, 19.0°, 22.2° and 24.8°±0.2° 2θ.

5. The crystalline Fluticasone furoate of claim 4, characterized by a PXRD pattern as depicted in the diffractogram of FIG. 1.

6. The crystalline Fluticasone furoate of claim 5, characterized by powder XRD peaks at 9.0°, 10.7°, 14.5°, 15.2° and 16.2°±0.2° 2θ.

7. The crystalline Fluticasone furoate of claim 5, wherein the crystalline Fluticasone furoate is a (S)-2-butanol solvate.

8. The crystalline Fluticasone furoate of claim 1, characterized by the following unit cell parameters: a 12.3686(6) Å, b=15.4737(5) Å, c=15.5235(5) Å, α=90 °, =90°, γ=90°, cell volume=2971.0(2) Å³, orthorhombic space group P2₁2₁2₁.

9. The crystalline Fluticasone furoate of claim 8, characterized by a crystal structure as shown in FIG. 4.

10. The crystalline Fluticasone furoate of claim 1, characterized by a solid state ¹³C NMR spectrum having peaks at 189.1, 165.5, 118.5 and 100.4±0.2 ppm.

11. The crystalline Fluticasone furoate of claim 10, characterized by a solid state ¹³C NMR spectrum as depicted in FIG. 2.

12. The crystalline Fluticasone furoate of claim 11 characterized by a solid-state ¹³C NMR spectrum having chemical shifts differences between the signal exhibiting the lowest chemical shift and another in the chemical shift range of 100 to 180 ppm of 88.7, 65.0 and 18.0±0.1 ppm.

13. The crystalline Fluticasone furoate of claim 11, wherein the crystalline Fluticasone furoate is a (S)-2-butanol solvate.

14. A crystalline form according to claim 1 for use as a pharmaceutical formulation.

15. A pharmaceutical composition comprising the crystalline Fluticasone furoate of claim 1 and at least one pharmaceutically acceptable excipient.
